(19) United States
(12) Patent Application Publication
(10) Pub. No.: US 2021/0308048 A1
(43) Pub. Date: Oct. 7, 2021

(54) EUTECTIC SOLVENTS COMPRISING PHARMACEUTICAL AGENTS, AND METHODS OF MAKING AND USE THEREOF

(71) Applicant: Novilla Pharmaceuticals, Inc., Arlington, VA (US)

(72) Inventor: Eric T. Fossel, Cambridge, MA (US)

(73) Assignee: Novilla Pharmaceuticals, Inc., Arlington, VA (US)

(21) Appl. No.: 17/264,739

(22) PCT Filed: Jul. 31, 2019
(86) PCT No.: PCT/US2019/044311

§ 371 Date: Jan. 29, 2021

Related U.S. Application Data
(60) Provisional application No. 62/817,065, filed on Mar. 12, 2019.
(60) Provisional application No. 62/817,071, filed on Mar. 12, 2019.
(60) Provisional application No. 62/791,110, filed on Jan. 11, 2019.
(60) Provisional application No. 62/778,954, filed on Dec. 13, 2018.
(60) Provisional application No. 62/778,949, filed on Dec. 13, 2018.
(60) Provisional application No. 62/713,022, filed on Aug. 1, 2018.

(51) Int. Cl. A61K 9/08; A61K 47/18; A61K 31/167; A61K 9/48; A61K 9/00; A61K 31/569; A61K 31/4545; A61K 31/495; A61K 38/28; A61K 31/4045

(52) U.S. Cl. CPC A61K 9/08; A61K 47/186; A61K 31/167; A61K 9/4858; A61K 9/0053; A61K 9/008; A61K 31/4545; A61K 31/495; A61K 38/28; A61K 31/4045; A61K 31/569

(57) ABSTRACT

The present invention generally relates to eutectic compositions comprising pharmaceutical agents and other beneficial substances, and in particular, to eutectic compositions where the pharmaceutical agent and beneficial substance forms a part of the eutectic composition. In one aspect, the pharmaceutical agent and beneficial substance defines part of the eutectic composition, i.e., rather than just being present within the eutectic composition. In one set of embodiments, at least about 20 mol % of the eutectic composition may comprise a hydrogen bond donor and a hydrogen bond acceptor, for example, acetaminophen and choline chloride, respectively. However, in other embodiments, the pharmaceutical agent can be either donor or acceptor, and the other agent either donor or acceptor. Other aspects are generally directed to methods of making such compositions, methods of using such compositions, kits including such compositions, etc.

### RELATED APPLICATIONS

This application claims the benefit of U.S. Provisional Patent Application Ser. No. 62/713,022, filed Aug. 1, 2018, entitled “Anhydrous Dosage Forms of Aspirin Based on Deep Eutectic Solvents and Other Specialized Media”; U.S. Provisional Patent Application Ser. No. 62/778,949, filed Dec. 13, 2018, entitled “Systems and Methods for Delivery of Drugs and Other Substances Comprising Deep Eutectic Solvents”; U.S. Provisional Patent Application Ser. No. 62/778,954, filed Dec. 13, 2018, entitled Methods and Systems Comprising Deep Eutectic Solvents for Poorly Soluble Drugs and Other Applications”; U.S. Provisional Patent Application Ser. No. 62/791,110, filed Jan. 11, 2019, entitled “Methods and Systems Comprising Deep Eutectic Solvents for Poorly Soluble Drugs and Other Applications”; U.S. Provisional Patent Application Ser. No. 62/817,065, filed Mar. 12, 2019, entitled “Eutectic Solvents Comprising Pharmaceutical Agents, and Methods of Making and Use Thereof”; and U.S. Provisional Patent Application Ser. No. 62/817,071, filed Mar. 12, 2019, entitled “Inhalable Eutectic Solvent Compositions and Methods.” Each of these is incorporated herein by reference in its entirety.

### FIELD

The present invention generally relates to eutectic compositions comprising pharmaceutical agents and other beneficial substances, and in particular, to eutectic compositions where the pharmaceutical agent and beneficial substance forms a part of the eutectic composition. In addition, some aspects generally relate to inhalable compositions, e.g., for the delivery of pharmaceutical agents and other beneficial substances. In some embodiments, such compositions may include eutectic solvents, including deep eutectic solvents.

### BACKGROUND

A eutectic is generally a mixture of substances that melts or solidifies at a single temperature that is lower than the melting point of either of the constituents. However, eutectics have not been commonly used for medical purposes.

### SUMMARY

The present invention generally relates to eutectic compositions comprising pharmaceutical agents and other beneficial substances, and in particular, to eutectic compositions where the pharmaceutical agent and beneficial substance forms a part of the eutectic composition. The subject matter of the present invention involves, in some cases, interrelated products, alternative solutions to a particular problem, and/or a plurality of different uses of one or more systems and/or articles.

In one aspect, the present invention is generally directed to a composition. In one set of embodiments, the composition comprises a eutectic, where at least 20 mol % of the eutectic comprises choline chloride and acetaminophen.

The composition, in another set of embodiments, comprises a eutectic, where at least 20 mol % of the eutectic comprises choline chloride and a pharmaceutical agent comprising a hydrogen bond donor.

The present invention, in another aspect, is generally directed to a method. In one set of embodiments, the method includes orally administering a composition to a subject, where the composition comprises a eutectic. In some cases, at least 20 mol % of the eutectic comprises choline chloride and acetaminophen.

The method, in another set of embodiments, includes orally administering a composition to a subject, where the composition comprises a eutectic composition. In some embodiments, at least 20 mol % of the eutectic composition comprises choline chloride and a pharmaceutical agent comprising a hydrogen bond donor.

In addition, some embodiments generally relate to inhalable compositions, e.g., for the delivery of pharmaceutical agents and other beneficial substances. For example, in one aspect, the present invention is generally directed to a method including administering a composition comprising a pharmaceutically acceptable deep eutectic solvent into the lungs of a subject. In some cases, the deep eutectic solvent comprises a pharmaceutical agent dissolved therein.

The present invention is generally directed to an article comprising an inhaler comprising a pharmaceutically acceptable deep eutectic solvent comprising a pharmaceutical agent, in another aspect.

In one embodiment, the present invention is generally directed to an inhalable composition comprising a deep eutectic mixture. The deep eutectic mixture may also comprise a pharmaceutical agent or other beneficial substance in some instances.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention encompasses methods of making one or more of the embodiments described herein, for example, a eutectic composition and/or an inhalable composition. In still another aspect, the present invention encompasses methods of using one or more of the embodiments described herein, for example, a eutectic composition and/or an inhalable composition.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying FIGURES, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a schematic eutectic diagram in accordance with certain embodiments of the invention.

### DETAILED DESCRIPTION

The present invention generally relates to eutectic compositions comprising pharmaceutical agents and other beneficial substances, and in particular, to eutectic compositions where the pharmaceutical agent and beneficial substance forms a part of the eutectic composition. In one aspect, the pharmaceutical agent and beneficial substance defines part of the eutectic composition, i.e., rather than just being present within the eutectic composition. In one set of embodiments, at least about 20 mol % of the eutectic composition may comprise a hydrogen bond donor and a hydrogen bond acceptor, for example, acetaminophen and choline chloride, respectively. However, in other embodiments, the pharmaceutical agent can be either donor or acceptor, and the other agent either donor or acceptor. Other aspects are generally directed to methods of making such compositions, methods of using such compositions, kits including such compositions, etc.

In addition, some embodiments of the present invention generally relate to inhalable compositions, e.g., for the delivery of pharmaceutical agents and other beneficial substances. For example, certain aspects are generally directed to compositions comprising eutectic solvents, including deep eutectic solvents. In some cases, such compositions may have surprisingly low melting points, for example, such that they are liquid at ambient temperatures. Such compositions, in some cases, may be useful for containing pharmaceutical agents or beneficial substances that are poorly soluble in water, or are sensitive to water, etc. Accordingly, such compositions may be liquid without necessarily being aqueous. In certain embodiments, such compositions can be administered to a subject, e.g., to the nose or lungs of a subject. For example, an inhaler can be used to administer various compositions. Other aspects are generally directed to methods of making such compositions, methods of using such compositions, kits including such compositions, etc.

In certain embodiments, two or more materials may be present within a composition that each have a melting point, but when mixed together, the resulting mixture may have a melting point that is lower than each of its component materials. Such a phenomenon is commonly referred to as a eutectic mixture, a eutectic solvent, or just a eutectic. In some cases, the melting point of the mixture may be lower than the melting points of the component materials. For example, the melting point may decrease by at least 10° C., at least 25° C., or at least 50° C. from the lowest of the component melting points. In some cases, the materials and their ratios are chosen such that the mixture is a liquid at room temperature, e.g., the mixture may have a melting point of less than 25° C., such that at ambient temperatures, the mixture is at a temperature above its melting point, and accordingly is liquid. In addition, in some cases, the mixture may be chosen such that it is a liquid at various temperatures, e.g., less than 20° C., less than 10° C., etc.

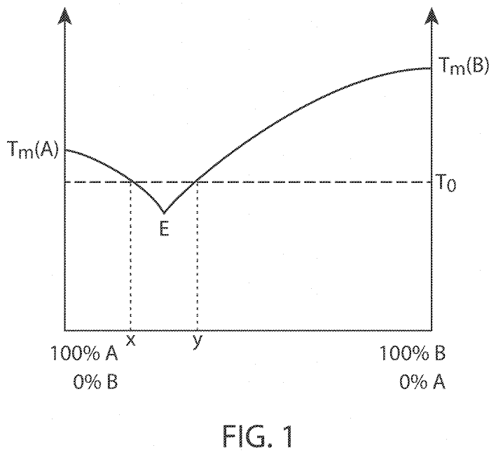

Although such a mixture of two or more materials may exhibit a lowest possible melting point at a specific ratio of materials (commonly referred to as the eutectic point or the eutectic ratio), such as is shown in FIG. 1, it should be understood that the invention is not limited to only those eutectic points or ratios, but instead also encompasses any mixture in which the melting point of the mixture is lower than each of its component materials, and typically where the mixture is liquid at ambient temperatures.

Accordingly, certain aspects of the invention are generally directed to eutectic where the pharmaceutical agent or beneficial substance forms an integral part of the eutectic. This is in contrast to compositions which are eutectic even in the absence of the pharmaceutical agent or beneficial substance. Thus, in some embodiments, a significant part of the eutectic may accordingly be a pharmaceutical agent or beneficial substance, for example, at least 25 mol % or at least 40 mol % of the eutectic may be a pharmaceutical agent.

In some embodiments, the eutectic may be a deep eutectic solvent. Deep eutectics can be formed from a mixture of a hydrogen bond donor, and a hydrogen bond acceptor, which form a eutectic liquid with a depressed melting point. For example, in some cases, a deep eutectic may be formed from a mixture of Lewis or Bronsted acids and bases. In addition, in some cases, the hydrogen bond donor may be a salt.

Thus, for example, in one set of embodiments, the eutectic may comprise a suitable salt and a pharmaceutical agent or a beneficial substance that includes a hydrogen bond acceptor. One non-limiting example is a mixture of choline chloride and acetaminophen (or APAP), which have the following structures, respectively:

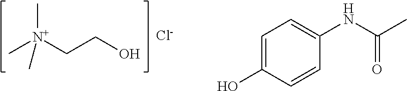

Without wishing to be bound by any theory, it is believed that the —N⁺ on choline chloride can participate in hydrogen bonding to the hydroxyl group on the benzene ring of acetaminophen, thereby allowing the combination to act as a eutectic.

The use of a eutectic may allow the administration of pharmaceutical agents (or other beneficial substances) that are relatively sensitive to water. For instance, the eutectic may be liquid, but nonetheless contain relatively low amounts of water. In addition, as the pharmaceutical agent or beneficial substance forms part of the eutectic, in some embodiments, relatively high amounts of the pharmaceutical agent or beneficial substance can be administered to the subject.

Accordingly, in one aspect, the present invention is generally directed to compositions, such as liquids, patches, creams, lotions, gels, or the like, that contain a eutectic containing a pharmaceutical agent or other beneficial substance. In some cases, the composition comprises or consists essentially of a eutectic, e.g., one that exhibits a lower melting point than the components forming the eutectic. Two, three, four, or more materials may be present that can be mixed together to form the eutectic. In some cases, the materials (when separate) are generally solid at ambient temperatures, but form a liquid when mixed together to form the eutectic.

In certain embodiments, two or more materials may be present within the eutectic mixture that each have a melting point, but when mixed together, the resulting mixture may have a melting point that is lower than each of its component materials. In some cases, the difference in melting point may be very large. For example, in some embodiments, the eutectic may exhibit a decrease by at least 10° C., at least 15° C., at least 20° C., at least 25° C., at least 30° C., at least 40° C., at least 50° C., at least 60° C., at least 70° C., at least 80° C., at least 90° C., or ° C., at least 100° C. In addition, in some embodiments, the melting point may be decreased sufficiently so that the eutectic is a liquid at room temperature, e.g., below 25° C. or 20° C. In certain embodiments, the eutectic may be liquid at temperatures of below 15° C., 10° C., 5° C., or 0° C.

One example of a eutectic mixture is urea and choline chloride. Other examples of eutectic mixtures include, but are not limited to, phenol/menthol, phenol/choline chloride, phenol/choline chloride/urea, betaine hydrochloride/urea, resorcinol/choline chloride, BHT/choline chloride, chloroxylenol/choline chloride/menthol, choline chloride/citric acid, choline chloride/arginine/urea, choline chloride/niacinamide/urea, camphor/menthol, camphor/menthol/lauryl alcohol, camphor/glycerin/monolaurate/menthol, etc. Additional non-limiting examples include EtNH₃Cl/CF₃CONH₂, EtNH₃Cl/acetamide, EtNH₃Cl/urea, ChCl/CF₃CONH₂, AcChCl/urea, ZnCl₂/urea, ZnCl₂/acetamide, ZnCl₂/ethylene glycol, ZnCl₂/hexanediol, ChCl/glycerol, ChCl/ethylene glycol, ChCl/malonic acid, Et₂(EtOH)NCl/glycerol, Et₂(EtOH)NCl/ethylene glycol, Me(PH)₃PBr/glycerol, Me(PH)₃PBr/ethylene glycol, ChCl/glucose, ChCl/1,4-butanediol, ChCl/CF₃CONH₂, ChCl/imidazole, ChCl/ZnCl₂, ChCl/xylitol, ChCl/sorbitol, ChCl/malonic acid, Bu₄NBr/imidazole, etc. Many of these materials are readily available commercially, and can be mixed together in any suitable ratio.

As mentioned, in some cases, the eutectic can be a deep eutectic. Such a eutectic may be formed from a mixture of a hydrogen bond donor, and a hydrogen bond acceptor. For instance a deep eutectic may be formed from a mixture of Lewis or Bronsted acids and bases. Either or both the hydrogen bond donor or the hydrogen bond acceptor may be a pharmaceutical agent or other beneficial substance. As a non-limiting example, a eutectic may be formed from choline chloride and acetaminophen, with the choline chloride acting as a hydrogen bond donor and the acetaminophen acting as a hydrogen bonda acceptor.

Accordingly, in one set of embodiments, a eutectic mixture comprises a first component containing a hydrogen bond donor, and a second component containing a hydrogen bond acceptor. The hydrogen bond donor, in some cases, may be a salt. In some cases, the hydrogen bond donor may contain an electronegative element, such as nitrogen or oxygen), bonded to a hydrogen atom (e.g., forming moieties such as —NH₂, —OH, or the like), in which case the hydrogen atom can participate in hydrogen boding to a suitable hydrogen bond acceptor.

One example of a hydrogen bond donor that can used in a eutectic is choline chloride. Other examples of suitable hydrogen bond donors include, but are not limited to, carnitine, acetyl carnitine, and guanidine. Still other examples of hydrogen bond donors include, but are not limited to, citric acid, levulenic acid, lactic acid, maleic acid, ketorolac, or the like. It should be understood that some species, such as ketorolac, cetirizine, or guanidine, may act as either a hydrogen bond acceptor or a hydrogen bond donor, depending on the environment. Specific non-limiting examples of combinations that can form eutectics include carnitine/acetaminophen or acetyl carnitine/acetaminophen.

According to some embodiments, the hydrogen bond donor may be pharmaceutically acceptable, or may be generally recognized as safe (for example, the components may be GRAS components as defined by the US FDA). In some cases, a pharmaceutically acceptable component is one that is generally safe, non-toxic and does not produce harmful or deleterious biological effects (e.g., at doses or amounts comparable to those that would expect typically given to a subject). This may include components acceptable for human or animal use. One example of a pharmaceutically acceptable hydrogen bond donor is choline chloride. Other non-limiting examples include those described above.

One example of a pharmaceutical agent that can act as a hydrogen bond acceptor is acetaminophen. Other examples of suitable pharmaceutical agents or other beneficial substances that can act as hydrogen bond acceptors include, but are not limited to those containing carboxyl groups, phenolic groups, or the like. Examples of pharmaceutical agents with carboxyl groups include, but are not limited to, ibuprofen, naproxen, diclofenac, and ketoprofen. Still other non-limiting examples include carnitine, acetyl carnitine, betaines such as glycine betaine, ketorolac, cetirizine, choline bitartrate, or the like. Examples of pharmaceutical agents with phenolic groups include, but are not limited to, steroids. Additional non-limiting examples include choline chloride or guanidine (e.g., guanidine HCl). It should be understood that in some cases, there is not a sharp distinction between a pharmaceutical agent and a beneficial substance, e.g., a pharmaceutical agent can also be a beneficial substance, e.g., a substance that is beneficial to the subject. Accordingly, in the descriptions herein, it should be understood that discussions of pharmaceutical agents are by way of example only, and in another embodiment, another beneficial substance may be present instead of (or in addition to) a pharmaceutical agent.

In some embodiments, the pharmaceutical agent or beneficial substance may include one or more groups which can serve as a hydrogen bond donor or hydrogen bond acceptor. In some cases, the pharmaceutical agent or beneficial substance may have a relatively small molecular weight (e.g., less than 1000 Da, less than 500 Da, less than 200 Da, or less than 100 Da), and may comprise a hydrogen bond donor or hydrogen bond acceptor.

In one set of embodiments, the pharmaceutical agent or beneficial substance may include small molecules (e.g., having a molecular weight of less than about 2,000 Da, less than about 1,500 Da, or less than about 1,000 Da), peptides (e.g., having less than about 10, less than about 15, less than about 20, or less than about 25 amino acids), proteins (typically larger than peptides), hormones, vitamins, nucleic acids, or the like.

It should be understood, as noted above, that a eutectic mixture need not have ratios of its component materials that produces the lowest possible melting point. Thus, the present invention is not limited to only eutectic ratios of components, but also includes, in other embodiments, other ratios able to cause decreases in the melting point. For example, with reference to FIG. 1 as a non-limiting schematic illustration, two components (“A”) and (“B”) may each exhibit certain melting points in isolation, but when A and B are mixed in various ratios (extending from 100% A on the left to 100% B on the right), the melting point of the components may decrease, e.g., to a point (the eutectic point, E) that is lower than the component melting points of A and B (Tₘ(A) and Tₘ(B). However, it should be understood that the invention is not limited to only that particular ratio of A and B which produces the lowest possible melting point, but also includes other ratios of A and B as well. For instance, a variety of different ratios of A and B may still produce a lower melting point than either A or B separately, even if other ratios of A and B may produce even lower melting point than that. For example, if melting points below some target temperature (To) is desired (e.g., ambient temperature), then any ratios of A and B between x and y would be suitable, not just the ratio at the eutectic point E. It should also be understood that FIG. 1 illustrates an idealized eutectic phase diagram (i.e., it is not to scale), and different eutectic components may exhibit different eutectic behavior, including more complex behaviors than is shown here.

In the eutectics mixtures disclosed above, and other eutectics described herein, each of the components may be present in any of a wide variety of ratios, e.g., such that the mixture exhibits a lower melting point than the components forming the mixture. In some cases, a first component (e.g., urea) may be present at between 5 mol % and 95 mol %, and the second component (e.g., choline chloride) may be present at between 5 mol % and 95 mol %. A third component (if present) may also be present at between 5 mol % and 95 mol %.

For example, a component may be present within the eutectic at at least 5 mol %, at least 10 mol %, at least 15 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, etc., and/or at no more than 95 mol %, no more than 90 mol %, no more than 85 mol %, no more than 80 mol %, no more than 75 mol %, no more than 70 mol %, no more than 65 mol %, no more than 60 mol %, no more than 55 mol %, no more than 50 mol %, no more than 45 mol %, no more than 40 mol %, no more than 35 mol %, no more than 30 mol %, no more than 25 mol %, no more than 20 mol %, no more than 15 mol %, no more than 10 mol %, etc. Combinations of any of these are also possible, e.g., a first component may be present at between 30 mol % and 40 mol %, between 25 mol % and 70 mol %, between 40 mol % and 60 mol %, between 60 mol % and 70 mol %, between 45 mol % and 55 mol %, etc. As other non-limiting examples, in some cases, the first component may each be present at between 25 mol % and 45 mol %, between 35 mol % and 45 mol %, between 30 mol % and 40 mol %, etc., and the second component may be present in these percentages, or in a different percentages.

However, it should be understood that in the eutectic mixture, these may sum to 100 mol %, or to other percentages in some cases, e.g., if other materials (such as pharmaceutical agents or beneficial substances) are also present. Thus, for example, the percentages of the first, second, third, etc., components may sum to at least 70 mol %, at least 75 mol %, at least 80 mol %, at least 85 mol %, at least 90 mol %, at least 95 mol %, at least 97 mol %, or at least 99 mol % of the eutectic, depending on other materials that might also be present. For example, in one set of embodiments, the eutectic-forming components may be present at at least 5 mol %, at least 10 mol %, at least 15 mol %, at least 20 mol %, at least 25 mol %, at least 30 mol %, at least 35 mol %, at least 40 mol %, at least 45 mol %, at least 50 mol %, at least 55 mol %, at least 60 mol %, at least 65 mol %, at least 70 mol %, at least 75 mol %, at least 80 mol %, at least 85 mol %, at least 90 mol %, etc., and/or at no more than 95 mol %, no more than 90 mol %, no more than 85 mol %, no more than 80 mol %, no more than 75 mol %, no more than 70 mol %, no more than 65 mol %, no more than 60 mol %, no more than 55 mol %, no more than 50 mol %, no more than 45 mol %, no more than 40 mol %, no more than 35 mol %, no more than 30 mol %, no more than 25 mol %, no more than 20 mol %, no more than 15 mol %, no more than 10 mol %, etc. Combinations of any of these are also possible, e.g., the eutectic-forming components may be present at between 20 mol % and 90 mol %, between 25 mol % and 40 mol %, or the like.

According to some embodiments, the components of the eutectic mixture may be pharmaceutically acceptable, or are generally recognized as safe (for example, the components may be GRAS components as defined by the US FDA). In some cases, a pharmaceutically acceptable component is one that is generally safe, non-toxic and does not produce harmful or deleterious biological effects (e.g., at doses or amounts comparable to those that would expect typically given to a subject). This may include components acceptable for human or animal use. One example of a pharmaceutically acceptable eutectic mixture is urea/choline chloride. Other non-limiting examples include choline chloride/arginine/urea, camphor/menthol, camphor/menthol/lauryl alcohol, ChCl/glycerol, and others including some of those described above.

Thus, it is important to note that certain embodiments of the invention are generally directed to eutectic mixtures containing non-toxic ingredients, such as a eutectic mixture of urea and choline chloride. Such non-toxic eutectic mixtures may not substantially deleteriously affect the subject, e.g., when applied to the nose or lungs of the subject, and accordingly can be used to deliver pharmaceutical agents or other beneficial substances, such as those described herein.

Accordingly, certain embodiments of the invention are generally directed to systems and methods for facilitating the absorption of pharmaceutical agents or beneficial substances that are poorly soluble in water. For example, a pharmaceutical agent or a beneficial substance may be contained within a eutectic mixture as described herein. In some cases, the eutectic mixture may not necessarily have a significant amount of water, e.g., the eutectic mixture may be substantially anhydrous, or have percentages of water such as those described herein. Accordingly, the solubility of the pharmaceutical agent or a beneficial substance in water is less of an issue in the eutectic mixture, e.g., the pharmaceutical agent or a beneficial substance may have a solubility within the eutectic mixture that is substantially different from its solubility in water. Thus, in certain cases, such eutectic mixtures may be used to deliver pharmaceutical agents or beneficial substances to a subject, e.g., by inhalation, or other techniques such as those described herein, without necessarily being limited to their water solubilities. Accordingly, even poorly soluble pharmaceutical agents or beneficial substances can be effectively administered.

As mentioned, in some cases, the composition may be substantially anhydrous. For example, the composition may contain less than 5%, less than 3%, less than 2%, less than 1%, less than 0.5%, less than 0.3%, less than 0.1%, less than 0.05%, less than 0.03%, less than 0.01%, less than 0.005%, less than 0.003%, or less than 0.001% water (by mole). In some cases, no detectable amounts of water may be present.

Different methods may be used to determine amount of water present, for example, FTIR, IR absorption, electrical resistivity measurements, or the like. As another example, in some embodiments, water may be detected by exposing the composition to a hydrolyzable agent (e.g., aspirin), and determining if the agent is hydrolyzed within the composition after a certain period of time (e.g., a day or a week).

In addition, as mentioned, other components may be added to the eutectic mixture. For example, in certain embodiments, the components may include one or more pharmaceutical agents. The composition may comprise one, two, three, or more pharmaceutical agents. One example is fluticasone. Other examples include loratadine or cetirizine. Another example is insulin. Yet another example is sumatriptan or other triptans. Still another example are the antihistamines. Still other examples include, but are not limited to, inhalational anesthetic agents such as aliflurane, chloroform, cyclopropane, desflurane, diethyl ether, halothane, isoflurane, methoxyflurane, methoxypropane, nitrous oxide, roflurane, sevoflurane, teflurane, trichloroethylene, vinyl ether, xenon, etc.; bronchodilators such as arformoterol, bitolterol, epinephrine, fenoterol, formoterol, ipratropium, isoetharine, isoproterenol, levalbuterol, metaproterenol, pirbuterol, procaterol, racepinephrine (racemic epinephrine), salbutamol, salmeterol, terbutaline, tiotropium, etc.; antihypertensives such as amyl nitrite, iloprost (prostacyclin), nitric oxide, etc.; anti-inflammatories such as beclomethasone, budesonide, ciclesonide, cromolyn, dexamethasone, flunisolide, fluticasone, mometasone, nedocromil, triamcinolone, etc.; antimicrobials such as pentamidine, ribavirin, tobramycin, zanamivir, etc.; pulmonary surfactants such as beractant, calfactant, colfosceril, poractant alfa, etc.; sympathomimetic amines such as amphetamine, levomethamphetamine, propylhexedrine, etc.; aromatic ammonia; dornase alfa; glutathione; insulin; methacholine; nicotine; or the like. Other examples include aspirin, caffeine, acetaminophen, niacinamide, naproxen, pseudoephedrine or other decongestants, phenethylamines, amphetamines, or the like. In some cases, a pharmaceutical agent may be present as a salt.

As another example, the component may include a beneficial substance. Non-limiting examples of beneficial substances include vitamins, cofactors, cosmetics, herbs, vitamins, minerals, dietary supplements, peptides, or the like. Non-limiting examples include coenzyme Q10, NAD (nicotinamide adenine dinucleotide), vitamin A, vitamin D, niacin, riboflavin, collagen, or the like. It should be understood that in some cases, there is not a sharp distinction between a pharmaceutical agent and a beneficial substance, e.g., a pharmaceutical agent can also be a beneficial substance, e.g., a substance that is beneficial to the subject. Accordingly, in the descriptions herein, it should be understood that discussions of pharmaceutical agents are by way of example only, and in another embodiment, another beneficial substance may be present instead of (or in addition to) a pharmaceutical agent.

As mentioned, one non-limiting example of a pharmaceutical agent is fluticasone. Fluticasone is an example of a glucocorticoid. A glucocorticoid is a class of steroid hormones that bind to the glucocorticoid receptor. Glucocorticoids are believed to exert their effects by binding to the glucocorticoid receptor (GR). The activated GR complex may up-regulate the expression of anti-inflammatory proteins in the nucleus (a process known as transactivation) and/or repress the expression of pro-inflammatory proteins in the cytosol by preventing the translocation of other transcription factors from the cytosol into the nucleus (transrepression). Thus, in general, glucocorticoids are believed to be part of the feedback mechanism in the immune system that decreases immune activity or inflammation. Glucocorticoids thus can be used to treat diseases or conditions caused by an overactive immune system, e.g., internally, such as allergies, asthma, autoimmune diseases, inflammatory diseases, autoinflammatory diseases, sepsis, psoriasis, arthritis, and the like. In addition, in some cases, a composition as is described herein can be used to treat pain, swelling, discomfort, and/or other symptoms caused by inflammation associated with a disease or condition in a joint, muscle or other target tissue. In some cases, the pain, swelling, discomfort, and/or other symptoms may be positioned within deeper tissues, rather than only locally or superficially.

Thus, in some embodiments, various aspects of the invention relate to compositions for delivering a glucocorticoid (such as fluticasone) or a salt, pro-drug, or derivative thereof to a subject. In some embodiments, the composition comprises a glucocorticoid or a salt, pro-drug, or derivative thereof. In certain embodiments, the composition further comprises one or more compounds that stabilize and/or otherwise promote the efficacy of storage and/or delivery.

The glucocorticoid may be naturally-occurring, or artificially synthesized. In one set of embodiments, the glucocorticoid may be a halogenated glucocorticoid, a fluorinated glucocorticoid, or a glucocorticoid having a thioester sidechain at C₁₇. Non-limiting examples of glucocorticoids that may be suitable for use as an anti-inflammatory include, but are not limited to, beclomethasone (pKa of 13-16), budesonide (pKa of 13-15), fluticasone (pKa of 12-14), mometasone (pKa of 12-13), and ciclesonide (pKa of 14-16). The structures of these compounds are respectively shown below:

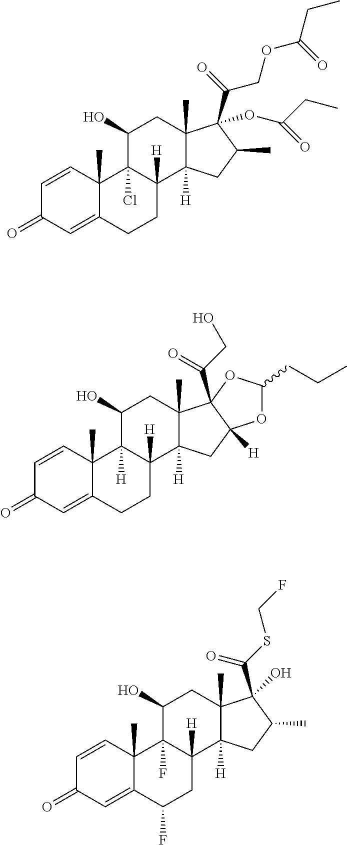

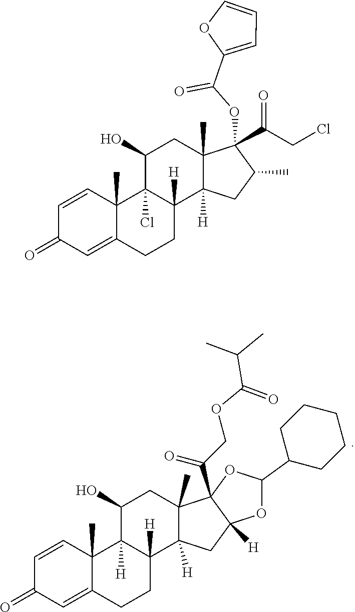

It should be appreciated that depending on the pKa of the glucocorticoid and/or the pH of the composition, the ionized form may be anionic or cationic (e.g., due to protonation). In some cases, the pH of the composition may be between about 3 and about 7, between about 3 and about 6, between about 3 and about 5, between about 4 and 8, between about 5 and about 8, between about 5 and 8.5, between about 7 and about 11, between about 8 and about 11, between about 9 and about 11, etc. Other pHs are described herein.

In addition, in some cases, the glucocorticoid may an ester, for example, an ester of one of the above compounds (where OR replaces one or more of the OH groups), or other compounds as discussed herein. Examples of such structures include, but are not limited to:

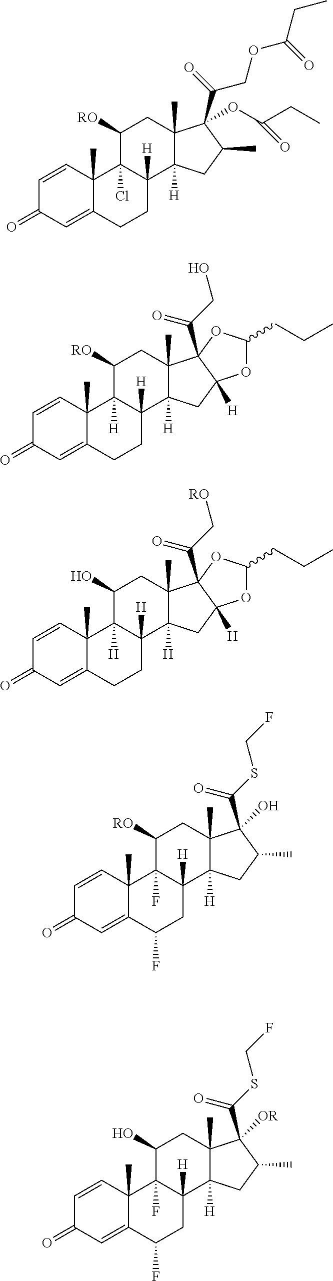

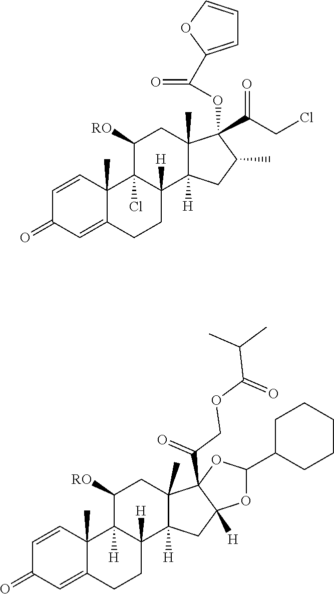

It should be noted that R in the above structures may be any suitable group, e.g., an alkyl group, which may be substituted or unsubstituted, and/or saturated (e.g., an alkenyl) or unsaturated. As non-limiting examples, the ester may be a propionate (where R is C₂H₅—CO—) or a furoate.

Accordingly, various embodiments of the invention are directed to compositions comprising glucocorticoids such as fluticasone, or salts or derivatives thereof for delivery to a subject, e.g., via inhalation. It should be understood that in any embodiment described herein using fluticasone, this is by way of example only, and other embodiments of the invention are directed to other glucocorticoids, salts of glucocorticoids, or derivatives of glucocorticoids, etc., instead of and/or in addition to fluticasone. The glucocorticoid may be any glucocorticoid described herein.

In another set of embodiments, the pharmaceutical agent is a triptan and/or a salt of a triptan. Their action is attributed to their binding to serotonin 5-HT1B and 5-HT1D receptors in cranial blood vessels (causing their constriction) and subsequent inhibition of pro-inflammatory neuropeptide release. These drugs may act on serotonin receptors in nerve endings as well as the blood vessels, which may lead to a decrease in the release of several peptides, including CGRP and substance P. Triptans generally have a structure:

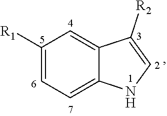

where R₁ may be a sulfonamide, a triazole (e.g., 1,2,3-triazole or 1,2,4-triazole), or a 2-oxazolidone; and R₂ may be a nitrogen-alkyl chain (e.g., —CH₂CH₂N(CH₃)₂), a dimethylpyrrolidine, or a 1-methyl-piperidine ring. A sulfonamide is generally a structure R^{a}SO₂NR^{b}R^{c}, where R^{a} may be an alkyl such as a C₁-C₅ alkyl (substituted or unsubstituted), for example, —CH₂—, CH₂CH₂—, —CH₂CH₂CH₂—, etc., and R^{b} and R^{c} may each independently be —H or an alkyl such as a C₁-C₅ alkyl (substituted or unsubstituted), for example, —CH₃, —CH₂CH₃, etc., or an aryl group (substituted or unsubstituted) such as phenyl.

Non-limiting examples of triptans include sumatriptan (pKas of 6.16, 9.63, and 17.14), rizatriptan, naratriptan (pKa of 17.11), zolmitriptan (pKa of 17.15), eletriptan, almotriptan (pKa of 8.77), frovatriptan (pKa of 17.27), and avitriptan (pKas of 3.6 and 8.0). The structures of these compounds are respectively shown below:

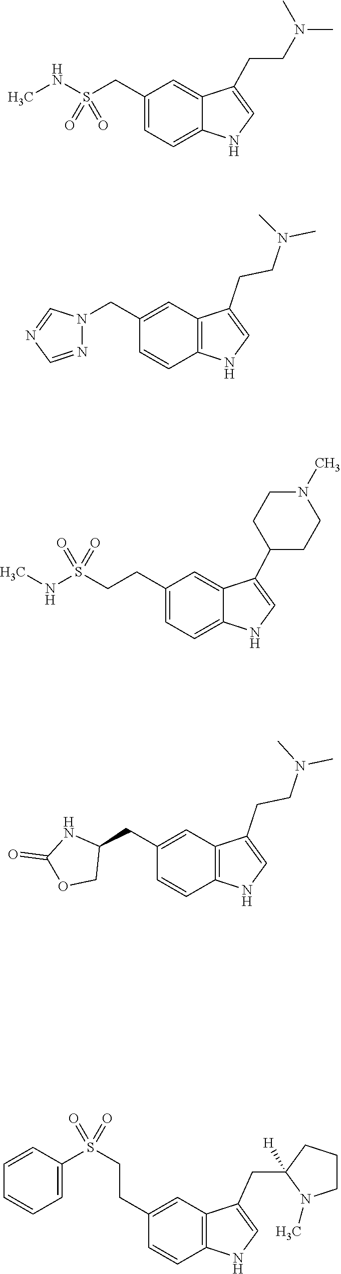

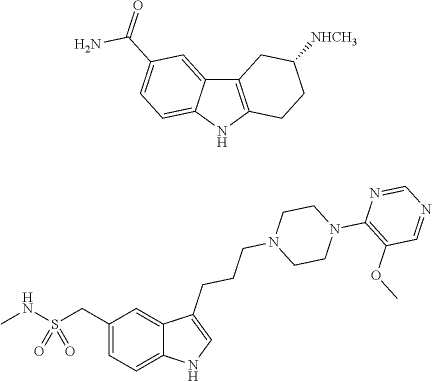 [SMILES: CN[C@@H]1CCc2[nH]c3ccc(C(N)=O)cc3c2C1 ; CNS(=O)(=O)Cc1ccc2[nH]cc(CCCN3CCN(c4ncncc4OC)CC3)c2c1]

In one set of embodiments, the Hi antihistamine may have a structure:

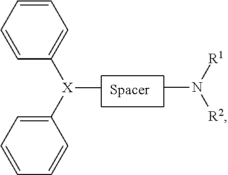

where X is CH, N, C(CH₃), or C(OH); the “Spacer” is usually 2-3 carbons in length, and may be linear, ring, branched, saturated or unsaturated; and R¹ and R² each independently may be —H, or a substituted or unsubstituted alkyl group, e.g., —CH₃. In some cases, X may be a chiral center. In certain cases, the two aromatic rings can be orientated in different planes; for example, the tricyclic ring system may be slightly puckered and the two aromatic rings may be arranged to lie in different geometrical planes.

Non-limiting examples of Hi antihistamines include, but are not limited to, fexofenadine (pKa of 13.20), cetirizine (pKa of 1.6-2.2, 2.9-3.0, 8.0-8.3) (or levocertrizine), clemastine, diphenhydramine (pKa of 8.2-9.1), doxylamine (pKa of 8.7-9.2), pheniramine (pKa of 4.2, 9.3-9.4), ebastine, chlorpheniramine (pKa of 9.2-9.4), meclizine, embramine, dexchlorpheniramine, and loratadine (pKa of 4.9-5.0). The structures of these compounds are respectively shown below:

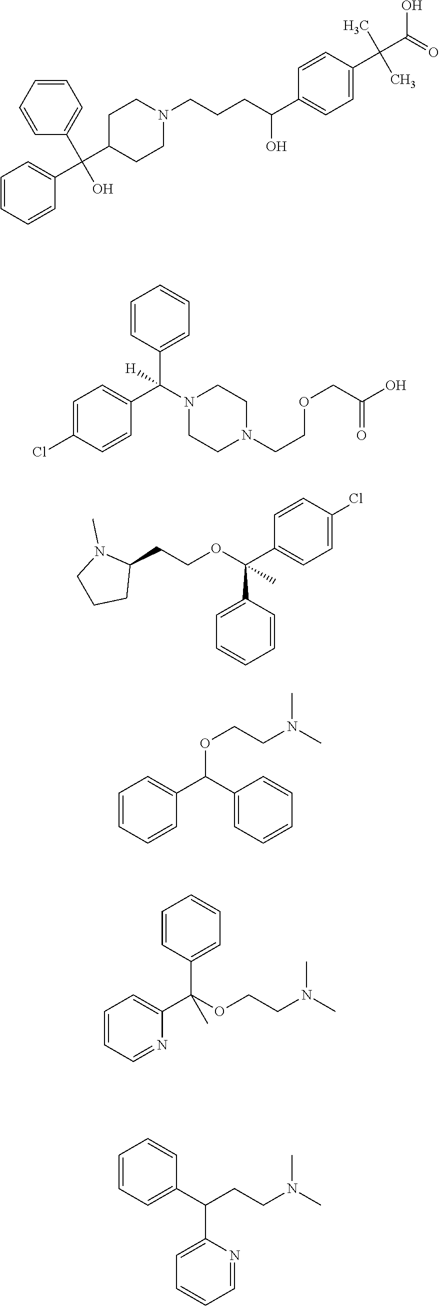 [SMILES: CC(C)(C(=O)O)c1ccc(C(O)CCCN2CCC(C(O)(c3ccccc3)c3ccccc3)CC2)cc1 ; O=C(O)COCCN1CCN([C@H](c2ccccc2)c2ccc(Cl)cc2)CC1 ; CN1CCC[C@@H]1CCO[C@](C)(c1ccccc1)c1ccc(Cl)cc1 ; CN(C)CCOC(c1ccccc1)c1ccccc1 ; CN(C)CCOC(C)(c1ccccc1)c1ccccn1 ; CN(C)CCC(c1ccccc1)c1ccccn1]

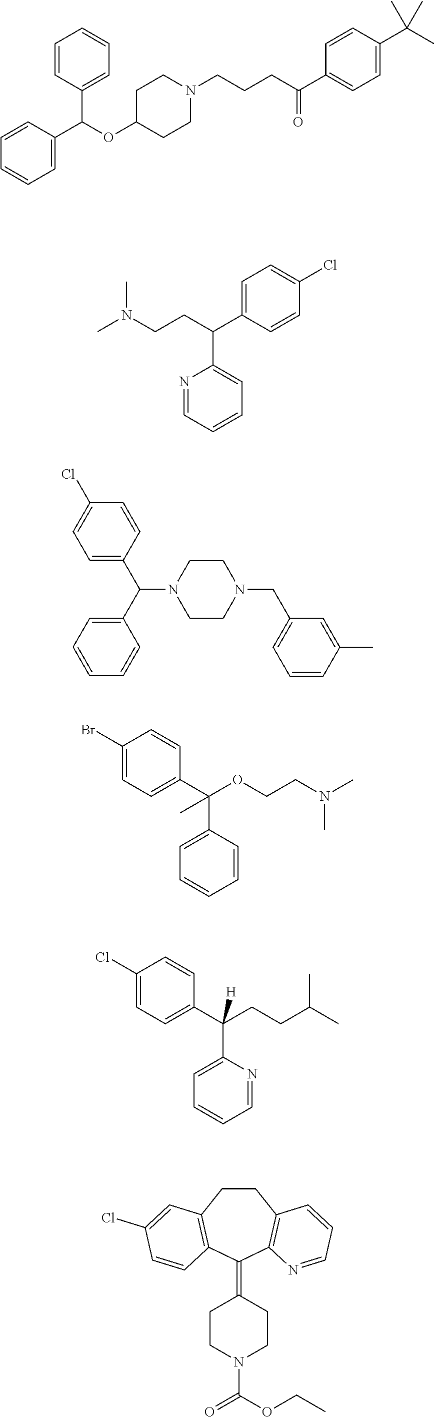

In some embodiments, the pharmaceutical agent or beneficial substance may be sensitive to water. For instance, a pharmaceutical agent or a beneficial substance may decompose or hydrolyze upon reaction with water. Without wishing to be bound by any theory, it is believed that in certain embodiments, such pharmaceutical agents or beneficial substances may be advantageously contained within a mixture, e.g., one that is substantially free of water. In some cases, the mixture may be a eutectic mixture, and in certain embodiments, one that is liquid at ambient temperatures. Such compositions may thus limit the amount of exposure of the pharmaceutical agents or beneficial substances to water. Thus, in some embodiments, a composition as described herein is substantially free of water. In some cases, the composition may contain less than 1 mol %, less than 0.01 mol %, or an undetectable amount of water. The composition may contain a pharmaceutical agent or a beneficial substance, for instance, one that can react with water.

In some cases, the pharmaceutical agents or beneficial substances may exhibit relative low solubilities in water. Without wishing to be bound by any theory, in some embodiments, such pharmaceutical agents or beneficial substances may be more soluble in mixtures such as those described herein. For instance, a pharmaceutical agent or a beneficial substance may be contained within a mixture that is substantially free of water. In some cases, the mixture may be an eutectic mixture, and in certain embodiments, one that is liquid at ambient temperatures. As examples, in certain embodiments, a pharmaceutical agent or a beneficial substance may have a solubility to water of less than 1000 mg/l, less than 500 mg/l, less than 300 mg/l, less than 100 mg/l, less than 50 mg/l, less than 30 mg/1, or less than 10 mg/1, etc.

Accordingly, some embodiments of the invention may be particularly advantages for the delivery of certain pharmaceutical agents, such as loratadine, that are insoluble or poorly soluble in water (i.e., aqueous solution). Such pharmaceutical agents can be dissolved within eutectic mixtures such as those described herein. For example, a composition may comprise a eutectic mixture, including pharmaceutical agents or other beneficial substances. The composition may, for example, be inhaled by a subject, for delivery to the lungs, or to the nasal tract, etc. For example, in some cases, the pharmaceutical agent or other beneficial substance may be dissolved within the eutectic mixture (i.e., “pre-dissolved”), and thus may be contained within the composition for delivery to the subject. Examples of pharmaceutical agents sensitive to water include, but are not limited to, loratadine, acetaminophen, or diphenhydramine.

In one set of embodiments, the first components and second components may be present in a mass ratio of between 2:1 and 1:2. For example, the ratio between the first component and the second component may be between 1.5:1 and 1:1.5, or between 1.2:1 and 1:1.2. In some cases, the mass ratio may be at least 1:2, at least 1:1.5, at least 1:1, at least 1.5:1, or at least 2:1, and/or no more than 2:1, no more than 1.5:1, no more than 1:1, no more than 1:1.5, or no more than 2:1. Of course, it should be understood that ratios outside these ranges are also possible in certain embodiments.

The pharmaceutical agents or beneficial substances may be present in any amount or concentration within the eutectic. For example, a pharmaceutical agent or a beneficial substance may be present within the eutectic at at least 0.01 mol %, at least 0.02 mol %, at least 0.03 mol %, at least 0.05 mol %, at least 0.1 mol %, at least 0.2 mol %, at least 0.3 mol %, at least 0.5 mol %, at least 1 mol %, at least 2 mol %, at least 3 mol %, at least 5 mol %, at least 10 mol %, at least 15 mol %, at least 20 mol %, at least 25 mol %, at least 30 mol %, at least 35 mol %, at least 40 mol %, at least 45 mol %, or at least 50 mol %, etc. In addition, in certain embodiments, the pharmaceutical agent or beneficial substance may be present within a composition at no more than 75 mol %, no more than 60 mol %, no more than 50 mol %, no more than 45 mol %, no more than 40 mol %, no more than 35 mol %, no more than 30 mol %, no more than 25 mol %, no more than 20 mol %, no more than 15 mol %, no more than 10 mol %, no more than 5 mol %, no more than 3 mol %, no more than 2 mol %, no more than 1 mol %, no more than 0.5 mol %, no more than 0.3 mol %, no more than 0.2 mol %, no more than 0.1 mol %, no more than 0.05 mol %, no more than 0.03 mol %, no more than 0.02 mol %, no more than 0.01 mol %, etc. Combinations of any of these ranges are also possible in various embodiments; for example, one (or more) of the pharmaceutical agent and/or beneficial substances may be present at between 40 mol % and 60 mol %, between 35 mol % and 50 mol %, between 1 mol % and 20 mol %, between 5 mol % and 20 mol %, between 5 mol % and 10 mol %, between 10 mol % and 25 mol %, between 0.5 mol % and 2 mol %, etc., of the composition. As a non-limiting example, fluticasone may be present at between 1 mol % and 5 mol %, between 1 mol % and 10 mol %, etc.

In some embodiments, a composition may comprise or consist essentially of a eutectic that comprises one or more pharmaceutical agents and/or beneficial substances that defines the eutectic. Water may or may not be present; if water is present, in certain embodiments, the amount of water that is present may be very low, e.g., less than 2 mol % or 1 mol %. Thus, the eutectic may be substantially anhydrous in some embodiments. In certain embodiments, the percentages of eutectic components (including pharmaceutical agents and/or beneficial substances), and water (if present) may sum to at least 50 mol %, at least 60 mol %, at least 70 mol %, at least 75 mol %, at least 80 mol %, at least 85 mol %, at least 90 mol %, at least 95 mol %, at least 97 mol %, or at least 99 mol %, or 100 mol % of the eutectic. However, it should be understood that low water contents are not required in all cases; in some embodiments, relatively high amounts of water may be present.

As a non-limiting example, in one set of embodiments, a composition may comprise urea and choline as eutectic components, and fluticasone as a pharmaceutical agent (alone, or with other pharmaceutical agents and/or other beneficial substances). For instance, the composition may comprise 5 mol % to 20 mol % of pharmaceutical agents and/or beneficial substances, 5 mol % to 95 mol % choline chloride, and 5 mol % to 95 mol % urea. Water may be present, or the composition may be substantially anhydrous. In some cases, water is present at less than 1 mol %. In some cases, the percentages of the pharmaceutical agents, choline chloride, urea, and water sum to at least 80 mol %, at least 90 mol %, or at least 100 mol % of the composition.

Such compositions may be prepared by any suitable technique. For example, in certain embodiments, a eutectic may be prepared by combining the two or more components of the eutectic together (e.g., as a mixture of solids), and supplying heat. Mixing the components to produce the eutectic may be endothermic in embodiments, such that some degree of heat may be needed to facilitate mixing and formation of the eutectic. For instance, the mixture may be heated to at least 30° C., at least 40° C., at least 50° C., at least 60° C., at least 70° C., at least 80° C., at least 90° C., at least 100° C., etc. In some cases, ambient temperatures may be sufficient to cause the eutectic to form. In certain embodiments, it may take at least 1 hour, at least 2 hours, at least 6 hours, at least 12 hours, at least 24 hours, etc. for the eutectic to form.

The above eutectics may be present within a composition in any suitable amounts. For example, one or more of the above eutectics may be present at at least about 0.1%, at least about 0.2%, at least about 0.3%, at least about 0.5%, at least about 1%, at least about 2%, at least about 3%, at least about 5%, at least about 10%, etc. In some cases, the eutectics may be present at no more than about 10%, no more than about 5%, no more than about 3%, no more than about 2%, no more than about 1%, no more than about 0.5%, no more than about 0.3%, no more than about 0.2%, or no more than about 0.1%. Combinations of any of these percentages are also possible. The actual concentration for a particular application can be determined by those of ordinary skill in the art using no more than routine experimentation, for example, by measuring the amount of transport of a compound as a function of concentration in vitro across cadaver skin or suitable animal models, skin grafts, synthetic model membranes, human models, or the like.

In addition, as mentioned, the eutectic may be substantially free of water in some embodiments. Such eutectics can be used, for example, in compositions such as liquids, patches, creams, lotions, gels, and the like. In some cases, the eutectic may contain less than 1 mol %, less than 0.01 mol %, or an undetectable amount of water. The eutectic may include a pharmaceutical agent or other beneficial substance, for instance, one that can react with water. In some cases, more than one pharmaceutically acceptable material may be present within the eutectic.

For example, certain embodiments of the invention are generally directed to systems and methods for facilitating the absorption of pharmaceutical agents or beneficial substances that are poorly soluble in water. For example, a pharmaceutical agent or a beneficial substance may be part of a eutectic mixture as described herein. In some cases, the eutectic may not necessarily have a significant amount of water, e.g., the eutectic may be substantially anhydrous, or have percentages of water such as those described herein. Accordingly, the solubility of the pharmaceutical agent or a beneficial substance in water is less of an issue in the eutectic. Thus, in certain cases, such eutectic mixtures may be used to deliver pharmaceutical agents or beneficial substances to a subject, e.g., orally, through the skin, or using other techniques such as those described herein, without necessarily being limited to their water solubilities. Accordingly, even poorly soluble pharmaceutical agents or beneficial substances can be effectively administered.

In one set of embodiments, the pharmaceutical agent or beneficial substance may be sensitive to water. For instance, a pharmaceutical agent or a beneficial substance may decompose or hydrolyze upon reaction with water. Without wishing to be bound by any theory, it is believed that in certain embodiments, such pharmaceutical agents or beneficial substances may be advantageously contained within a eutectic, e.g., one that is substantially free of water. In some cases, the eutectic may be liquid at ambient temperatures. Such eutectics may thus limit the amount of exposure of the pharmaceutical agents or beneficial substances to water.

In another set of embodiments, the pharmaceutical agents or beneficial substances may exhibit relative low solubilities in water. Without wishing to be bound by any theory, in some embodiments, such pharmaceutical agents or beneficial substances may be more soluble in eutectic mixtures such as those described herein. For instance, a pharmaceutical agent or a beneficial substance may be contained within a eutectic that is substantially free of water. As examples, in certain embodiments, a pharmaceutical agent or a beneficial substance may have a solubility to water of less than 1000 mg/l, less than 500 mg/l, less than 300 mg/l, less than 100 mg/l, less than 50 mg/l, less than 30 mg/1, or less than 10 mg/1, etc.

Thus, in some cases, the eutectic may be substantially anhydrous. For example, the eutectic may contain less than 5%, less than 3%, less than 2%, less than 1%, less than 0.5%, less than 0.3%, less than 0.1%, less than 0.05%, less than 0.03%, less than 0.01%, less than 0.005%, less than 0.003%, or less than 0.001% water (by mole). In some cases, no detectable amounts of water may be present.

Different methods may be used to determine amount of water present, for example, FTIR, IR absorption, electrical resistivity measurements, or the like. As another example, in some embodiments, water may be detected by exposing the eutectic to a hydrolyzable agent (e.g., aspirin), and determining if the agent is hydrolyzed within the composition after a certain period of time (e.g., a day or a week).

Some embodiments of the invention may be particularly advantages for the delivery of certain pharmaceutical agents, such as acetaminophen, using eutectics such as those described herein. For example, a composition such as a patch, cream, lotion, gel, or the like may comprise a eutectic, including pharmaceutical agents or other beneficial substances, e.g., as described herein. The composition may, for example, be applied to the skin of a subject, which may be used to deliver the pharmaceutical agent (or other beneficial substances) into the skin of the subject. For example, in some cases, the pharmaceutical agent or other beneficial substance may be dissolved within the eutectic mixture (i.e., “pre-dissolved”), and thus may be contained within the composition for delivery into the skin of the subject.

In some aspects, eutectic compositions such as those described above may be used in a suitable composition, such as a liquid, a patch, a cream, a lotion, a gel, or the like. For example, as discussed herein, components that are sensitive to water and/or are not easily soluble in water may be present in such compositions.

For instance, in one embodiment, a eutectic mixture may be mixed with a suitable adhesive compound and used to make a patch, or a bandage. As further examples, a eutectic such as described herein may be formed within creams, lotions, gels, or other compositions. In some cases, as discussed herein, such compositions may be substantially anhydrous. For example, such compositions may have water contents of less than 5 wt %, less than 2 wt %, or less than 1 wt %, or other water concentrations such as those discussed herein. In some cases, the transdermal patch or bandage may comprise a backing layer and an adhesive. The adhesive may in some cases contain a pharmaceutical agent (or other beneficial substance), or there may be a separate layer or portion of the patch or bandage that contains the pharmaceutical agent or other beneficial substance. Those of ordinary skill in the art will be familiar with transdermal patches, bandages, and the like.

For example, non-limiting examples of adhesives include, but are not limited to polyacrylate polymers, rubber-based adhesives and polysiloxane adhesives, natural or synthetic polyisoprene, polybutylene, polyisobutylene, styrene-butadiene polymers, styrene based polymers, styrene block copolymers, butadiene based polymers, styrene/butadiene polymers, styrene-isoprene-styrene block copolymers, hydrocarbon polymers, such as butyl rubber, halogen-containing polymers such as, for example, polyacrylonitrile, polytetrafluoroethylene, polyvinylchloride, polyvinylidene chloride, and polychlorodieneas polyisobutylenes, polybutylenes, ethylene/vinyl acetate and vinyl acetate based adhesives, styrene/butadiene adhesives, etc.

In one set of embodiments, the eutectic mixture is used within an emulsion, e.g., to form a cream, lotion, or other suitable composition. As known by those of ordinary skill in the art, an emulsion typically includes a first phase (e.g., a discontinuous phase) contained within a second fluid phase (e.g., a continuous phase). The eutectic may be present in either or both phases. In addition, other materials such as those described herein may be present in the same phase as the eutectic. In some embodiments, the emulsion may take the form of a cream or a lotion. For example, a eutectic may be contained within a hydrophobic, oily environment such as in an oil-based cream or lotion containing little or no water.

As an example, a cream may include materials such as oils, triglycerides, stearates, fatty acids, fatty alcohols, squalenes, polysorbates, or the like. In some cases, such materials are hydrophobic, which can be emulsified with water or other aqueous phases, e.g., to produce an emulsion. In one embodiment, for example, the cream may include a saturated squalene. Examples of stearates include, but are not limited to, glyceryl stearate, propylene glycol stearate, steryl stearate, sorbitan stearate, sodium stearate, calcium stearate, magnesium steratae, glycol sterate, and the like. Non-limiting examples of oils include mineral oil, wheat germ oil, palm oil, nut oil, linseed oil, etc. Other materials may also be present within the composition, for example, buffers, preservatives, surfactants, etc.

In one set of embodiments, as a non-limiting example, a cream may include one or more of water, mineral oil, glyceryl stereate, squalene, propylene glycol stearate, wheat germ oil, glyceryl stearate, isopropyl myristate, steryl stearate, polysorbate 60, propylene glycol, oleic acid, tocopherol acetate, collagen, sorbitan stearate, vitamin A and D, triethanolamine, methylparaben, aloe vera extract, imidazolidinyl urea, propylparaben, PND, and/or BHA. As another example, a cream may include one or more of water, sodium chloride, potassium chloride, L-arginine HCl, mineral oil, caprylic/capric triglycerides, phenoxyethanol, glycerol stearate, PEG 75 stearate, cetyl alcohol, methylparaben, and propylparaben.

In addition, in certain aspects, the present invention is generally directed to systems and methods for administering inhalable compositions to a subject, for example, for delivery to the nose or lungs of the subject. Such compositions, in some embodiments, may include eutectic solvents, such as deep eutectic solvents. These may have surprisingly low melting points, for example, such that they are liquid at ambient temperatures, and accordingly can be aerosolized or formed into droplets for inhalation. The compositions may also include pharmaceutical agents or beneficial substances that are poorly soluble in water, or are sensitive to water, etc., as although the eutectic is liquid, it is not necessarily aqueous. Pharmaceutical agents or beneficial substances can be dissolved within the eutectic solvent, and accordingly delivered as an inhalable composition. In contrast, many prior art inhalable compositions are based on water, in which such pharmaceutical agents or beneficial substances may be present in low concentrations, or precipitated in solid form, resulting in poor inhalation delivery to the subject.

Such inhalable compositions, in accordance with one aspect, may be delivered to a subject using an inhaler or other device able to cause delivery of the composition to the lungs of the subject. A variety of pharmaceutical agents can be delivered, including fluticasone and other agents discussed below. For administration by inhalation, the compositions may be conveniently delivered, for example, in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges for use in an inhaler may be used in some cases, and may contain a composition such as is described herein.

Also contemplated herein is pulmonary delivery of certain compositions as discussed herein. The compositions may be delivered to the lungs of a mammal while inhaling and delivered to the lung epithelial lining, e.g., in order to access the blood stream. In addition, contemplated for use in some embodiments of the invention are a wide range of mechanical devices designed for pulmonary delivery of therapeutic products, including but not limited to nebulizers, metered dose inhalers, and inhalers, all of which are familiar to those skilled in the art. Some non-limiting specific examples of commercially available devices are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler inhaler, manufactured by Fisons Corp., Bedford, Mass.

In some cases, a composition may be specific to the type of device employed and may involve the use of an appropriate propellant material, in addition to materials such as diluents, adjuvants, carriers, etc., useful in therapy, including those described herein. Also, the use of liposomes, microcapsules or microspheres, inclusion complexes, or other types of carriers is contemplated in certain embodiments of the invention. Chemically modified systems may also be prepared for use in different compositions depending on the type of chemical modification or the type of device employed, in certain embodiments of the invention.

Compositions suitable for use with a nebulizer, e.g., jet or ultrasonic, may include a buffer and a simple sugar (e.g., for stabilization of the composition and regulation of osmotic pressure). The nebulizer composition may also contain, in some cases, a surfactant to reduce or prevent surface induced aggregation caused by atomization of the solution in forming the aerosol.

Compositions for use with a metered-dose inhaler device may generally comprise a composition as described herein. In some cases, these may be propelled using a propellant, optionally with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Nasal delivery of a composition such as is discussed herein is also contemplated. Nasal delivery allows the passage of a composition to the blood stream directly after administering the therapeutic product to the nose, without the necessity for deposition in the lung. Compositions for nasal delivery may include those with dextran or cyclodextran.

For nasal administration, a useful device is a small, hard bottle to which a metered dose sprayer is attached. In one embodiment, the metered dose is delivered by drawing a composition into a chamber of defined volume, which chamber has an aperture dimensioned to aerosolize and aerosol composition by forming a spray when a liquid in the chamber is compressed. The chamber may be compressed to administer the composition. In a specific non-limiting embodiment, the chamber is a piston arrangement. Such devices are commercially available.

In another embodiment, a plastic squeeze bottle with an aperture or opening dimensioned to aerosolize an aerosol composition by forming a spray when squeezed is used. The opening is usually found in the top of the bottle, and the top is generally tapered to partially fit in the nasal passages for efficient administration of the aerosol composition. In some cases, the nasal inhaler will provide a metered amount of the aerosol composition, for administration of a measured dose of the drug.

In addition, some aspects of the invention are generally directed to inhalable compositions, e.g., comprising a eutectic mixture. The eutectic mixture may also comprise a pharmaceutical agent or other beneficial substance. In certain embodiments, two or more materials may be present within a composition that each have a melting point, but when mixed together, the resulting mixture may have a melting point that is lower than each of its component materials. Such a phenomenon is commonly referred to as a eutectic mixture, a eutectic solvent, or just a eutectic. In some cases, the melting point of the mixture may be lower than the melting points of the component materials. For example, the melting point may decrease by at least 10° C., at least 25° C., or at least 50° C. from the lowest of the component melting points. In some cases, the materials and their ratios are chosen such that the mixture is a liquid at room temperature, e.g., the mixture may have a melting point of less than 25° C., such that at ambient temperatures, the mixture is at a temperature above its melting point, and accordingly is liquid. In addition, in some cases, the mixture may be chosen such that it is a liquid at various temperatures, e.g., less than 20° C., less than 10° C., etc. However, it should be understood that the mixture may not necessarily be liquid at room temperature. For instance, in some cases, the mixture may have a melting point of less than 60° C., less than 55° C., less than 50° C., less than 45° C., less than 40° C., less than 35° C., less than 30° C., etc., but greater than room temperature (about 25° C.).

Although such a mixture of two or more materials may exhibit a lowest possible melting point at a specific ratio of materials (commonly referred to as the eutectic point or the eutectic ratio), such as is shown in FIG. 1, it should be understood that the invention is not limited to only those eutectic points or ratios, but instead also encompasses any mixture in which the melting point of the mixture is lower than each of its component materials, and typically where the mixture is liquid at ambient temperatures.

In some cases, the eutectic mixture is a deep eutectic mixture, which can be formed from a mixture of Lewis or Bronsted acids and bases. One example is choline chloride and urea. These can be mixed in a mole ratio of 1:2 to produce a eutectic mixture with a melting point of 12° C. However, as noted above, in other embodiments, other mole ratios may also be used to produce mixtures having lowered melting points, e.g., that are less than ambient temperatures. In addition, other examples of eutectic mixtures are discussed in more detail herein.

Accordingly, in one aspect, the present invention is generally directed to compositions that may be delivered to a subject by inhalation, e.g., for nasal delivery, pulmonary delivery, mucosal delivery, or the like. In some cases, the composition comprises or consists essentially of a eutectic, e.g., one that exhibits a lower melting point than the components forming the eutectic. Two, three, four, or more materials may be present that can be mixed together to form the eutectic. In some cases, the materials (when separate) are generally solid at ambient temperatures, but form a liquid when mixed together to form the eutectic.

In another aspect, a composition as described herein may be administered to a subject, either by itself and/or in conjunction with co-factors, other therapeutics, or the like. In some cases, the composition includes a pharmaceutically acceptable eutectic mixture, e.g., as described herein. In some embodiments, the composition may be applied using an inhaler. In certain embodiments, the composition may be applied in the form of a patch, cream, lotion, or gel, or the like, such as described herein. For example, a composition may be administered alone, or in conjunction with other compositions. When administered, the compositions can be applied in a therapeutically effective, pharmaceutically acceptable amount as a pharmaceutically acceptable formulation, for example, a pharmaceutically acceptable carrier such as those described below. The term “effective amount” of a composition, such as those described herein, refers to the amount necessary or sufficient to realize a desired biologic effect. As examples, an effective amount of acetaminophen to treat pain may be an amount sufficient to alleviate or reduce the sensation of pain, or an effective amount of fluticasone may be an amount sufficient to alleviate or reduce symptoms of asthma. In some cases, at least some of the pain may be subjective. Combined with the teachings provided herein, by choosing among the various active compositions and weighing factors such as potency, relative bioavailability, patient body weight, severity of adverse side effects and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular compositions being administered the size of the subject, or the severity of the disease or condition. One of ordinary skill in the art can empirically determine the effective amount of the compositions without necessitating undue experimentation.

The terms “treat,” “treated,” “treating,” and the like, when used herein, refer to administration of the compositions to a subject which may increase the resistance of the subject to development or further development of the disease or condition, to administration of the composition after the subject has developed the disease or condition in order to eliminate or at least control development of the disease or condition, and/or slow the progression of or to reduce the severity of symptoms caused by the disease or condition. When administered to a subject, effective amounts may depend on the particular disease or condition being treated and the desired outcome. A therapeutically effective dose may be determined by those of ordinary skill in the art, for instance, employing factors such as those further described below and using no more than routine experimentation.

For use in therapy, an effective amount of the compositions can be administered to a subject by any mode that delivers the composition to the subject, e.g., oral, pulmonary, topical, transdermal, or the like. As an example, a eutectic mixture, such as a deep eutectic mixture, may be contained within an inhaler that can be administered to the subject.

In some cases, a liquid, gel, or the like, such as is described herein, may be contained within a capsule that can be orally administered to a subject. For instance, in one set of embodiments, a eutectic mixture may be contained within a capsule, such as a hard capsule or a soft capsule. The capsule may comprise, for instance, gelatin, hypromellose, pullulan, carrageenans, starch, cellulose, or other materials known to those of ordinary skill in the art.

Accordingly, certain embodiments may generally be directed to a capsule or other vehicle containing a eutectic such as discussed herein. Examples of vehicles include, but are not limited to, capsules, tablets, pills, or the like. For example, the eutectic may be a eutectic containing APAP or acetaminophen. Non-limiting examples of such eutectics include choline chloride/acetaminophen, carnitine/acetaminophen, acetyl carnitine/acetaminophen, or other eutectics such as those described herein. In some cases, surprisingly, such vehicles may be smaller than commercially-available vehicles used for administering acetaminophen, e.g., orally, to a subject.

For instance, a capsule or other vehicle such as discussed herein may have contain at least 100 mg, at least 200 mg, at least 300 mg, at least 400 mg, at least 500 mg, at least 600 mg, at least 700 mg, at least 800 mg, at least 900 mg, at least 1000 mg, etc. of a drug such as acetaminophen, or others including those described herein. In some cases, the capsule or other vehicle may contain no more than 1000 mg, no more than 900 mg, no more than 800 mg, no more than 750 mg, no more than 700 mg, no more than 650 mg, no more than 600 mg, no more than 550 mg, no more than 500 mg, no more than 400 mg, no more than 300 mg, no more than 200 mg, or no more than 100 mg of the drug. As a non-limiting example, a capsule or other vehicle may contain about 500 mg of acetaminophen, e.g., between 400 mg and 600 mg.

In some cases, the capsule or other vehicle may have a size and/or volume that is relatively easy to swallow. For example, the capsule or other vehicle may have a maximum dimension of less than 20 mm, less than 19 mm, less than 18 mm, less than 17 mm, less than 16 mm, less than 15 mm, less than 14 mm, less than 13 mm, less than 12 mm, less than 11 mm, less than 10 mm, less than 9 mm, less than 8 mm, less than 7 mm, less than 6 mm, less than 5 mm, etc. The capsule may have a volume of, for example, less than 1.5 ml, less than 1.3 ml, less than 1.2 ml, less than 1.1 ml, less than 1.0 ml, less than 0.9 ml, less than 0.8 ml, less than 0.7 ml, less than 0.6 ml, less than 0.5 ml, less than 0.4 ml, less than 0.3 ml, etc. The capsule or other vehicle may be round or oblong, or other shapes in some cases. In some cases, a vehicle such as a capsule may have a hard shell or a soft shell, and/or be coated or uncoated.

In some cases, vehicles containing a higher concentration of a drug such as acetaminophen may be achievable, for example, than those that are commercially available. Without wishing to be bound by any theory, it is believed that such higher concentrations may be achieved since no additional solvents are needed, e.g., as the drug may form part of the eutectic mixture, as discussed herein. Thus, for example, the concentration of drug within a capsule (or other vehicle) may be at least 10%, at least 15%, at least 20%, or at least 25% higher than similar capsules containing a solvent, e.g., as obtainable commercially. In some cases, the capsule may have a volume that is at least 10%, at least 15%, at least 20%, or at least 25% smaller than similar capsules containing a solvent, e.g., the volume of the capsule may be less than 90%, less than 85%, less than 80%, less than 75%, less than 70%, less than 65%, or less than 60% of a similar capsule containing a solvent.

Thus, certain embodiments of the invention are generally directed to unexpectedly high concentrations of certain drugs such as acetaminophen. Such concentrations can be achieved using eutectic solvents, including deep eutectic solvents such as those described herein. For instance, as noted above, a drug such as acetaminophen may act as a component of the eutectic, thereby reducing or eliminating the need for additional components (such as solvents), which may result in much lower concentrations of drugs.

For instance, in one set of embodiments, the concentration (or the density) of the eutectic (e.g., a drug such as acetaminophen and another component, such as choline chloride) may be at least 1.2 g/ml, at least 1.25 g/ml, at least 1.3 g/ml, at least 1.35 g/ml, at least 1.4 g/ml, at least 1.45 g/ml, at least 1.5 g/ml, at least 1.6 g/ml, at least 1.7 g/ml, at least 1.8 g/ml, at least 1.9 g/ml, or at least 2 g/ml of drug within a eutectic composition and/or within a capsule or other vehicle such as is described herein. In some cases, the concentration may be no more than 2 g/ml, no more than 1.9 g/ml, no more than 1.8 g/ml, no more than 1.7 g/ml, no more than 1.6 g/ml, no more than 1.5 g/ml, no more than 1.48 g/ml, no more than 1.45 g/ml, no more than 1.4 g/ml, no more than 1.35 g/ml, or no more than 1.3 g/ml. Combinations of any of these are possible. For instance, the concentration may be between 1.25 and 1.48 g/ml, according to one embodiment. Such higher concentrations may result in much smaller volumes, e.g., as discussed above. As a non-limiting example, 500 mg of acetaminophen at a concentration of 1.48 g/ml could be achieved in a volume of 0.34 ml.

Administering the pharmaceutical composition may be accomplished by any method. In administering the compositions to a subject, dosing amounts, dosing schedules, routes of administration, and the like may be selected so as to affect known activities of these compositions. Dosages may be estimated based on the results of experimental models, optionally in combination with the results of assays of compositions described herein. Dosage may be adjusted appropriately to achieve desired drug levels, local or systemic, depending upon the mode of administration. The doses may be given in one or several administrations per day. Multiple doses per day are also contemplated in some cases to achieve appropriate systemic levels of the compositions within the subject.

The dose of the compositions to the subject may be such that a therapeutically effective amount of the compositions reaches the subject. The dosage may be given in some cases at the maximum amount while avoiding or minimizing any potentially detrimental side effects within the subject. The dosage of the compositions actually administered may be dependent upon factors such as the final desired concentration, the method of administration to the subject, the efficacy of the composition, the longevity of the composition within the subject, the timing of administration, the effect of concurrent treatments, etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition. Variations in dosing may occur between different individuals or even within the same individual on different days. In some cases, a maximum dose be used, that is, the highest safe dose according to sound medical judgment. In some cases, the dosage form is such that it does not substantially deleteriously affect the subject.

Subject doses of the compositions described herein for delivery may range from about 0.1 microgram to 10 mg per administration, which depending on the application could be given daily, weekly, or monthly and any other amount of time therebetween. In some cases, doses range from about 10 microgram to 5 mg per administration, e.g., from about 100 microgram to 1 mg, with 2 to 4 administrations being spaced days or weeks apart. In some embodiments, doses range from 1 microgram to 10 mg per administration, and most typically 10 microgram to 1 mg, with daily or weekly administrations. Other suitable dosings have been described in detail herein.

The compositions may be administered in multiple doses over extended period of time. For any composition described herein the therapeutically effective amount can be initially determined from animal models. The applied dose can be adjusted based on the relative bioavailability and potency of the administered pharmaceutical agent or other beneficial substance. Adjusting the dose to achieve maximal efficacy based on the methods described above and other methods as are well-known in the art is well within the capabilities of the ordinarily skilled artisan.

The treatments disclosed herein may be given to any subject, for example, a human, or a non-human animal, such as a dog, a cat, a horse, a rabbit, a cow, a pig, a sheep, a goat, a rat (e.g., Rattus Norvegicus), a mouse (e.g., Mus musculus), a guinea pig, a non-human primate (e.g., a monkey, a chimpanzee, a baboon, an ape, a gorilla, etc.), or the like.

Administration of a composition of the invention may be accomplished by any medically acceptable method. The particular mode selected may depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the compositions within the subject without causing clinically unacceptable adverse effects.

In some aspects, a composition as discussed herein is administered to a subject. Such administration may be systemic or localized, e.g., directed to a specific location of the body of a subject. A composition as discussed herein may be applied to a subject in some embodiments, such as a human subject. The composition may be applied in any suitable form, e.g., as discussed herein. For example, the composition may be applied using a delivery vehicle such as a cream, gel, liquid, lotion, spray, aerosol, or transdermal patch. In one set of embodiments, a composition may be applied or impregnated in a bandage or a patch applied to the skin of a subject. In some embodiments, a patch has a skin contacting portion made of any suitable material that is covered or impregnated with a cream or emulsion described herein, wherein the skin contacting portion may be supported by a backing, one or both of which may have an adhesive segment or other configuration for attaching to the skin surface of a subject.

Such compositions may be applied to the skin of a subject, such as a human subject. Examples of compositions are discussed herein. The composition may promote transfer into the skin of an effective concentration of the pharmaceutical agent or beneficial substance, directly or indirectly. For instance, the composition may include one or more penetrating agents, as further described herein. Those of ordinary skill in the art will know of systems and techniques for incorporating a nitric oxide donor and/or a pharmaceutical agent within delivery vehicles such as a cream, gel, lotion, spray, aerosol, or transdermal patch.

Thus, as another example, the composition may be deliver to the lungs, nose, etc. of an effective concentration of the pharmaceutical agent or beneficial substance, directly or indirectly. Those of ordinary skill in the art will know of systems and techniques for incorporating a pharmaceutical agent or a beneficial substance, e.g., within delivery vehicles such as liquids or aerosols.

In certain embodiments of the invention, the administration of a composition as described herein may be designed so as to result in sequential exposures to the composition over a certain time period, for example, hours, days, weeks, months, or years. This may be accomplished, for example, by repeated administrations of the composition by one of the methods described herein, or by a sustained or controlled release delivery system in which a composition is delivered over a prolonged period without repeated administrations. Administration of a composition using such a delivery system may be, for example, by methods such as those described herein. Maintaining a substantially constant concentration of a composition may be desired in some cases.

In certain embodiments of the invention, a composition can be combined with a suitable pharmaceutically acceptable carrier, for example, such as those described herein. In general, pharmaceutically acceptable carriers suitable for use are well-known to those of ordinary skill in the art. As used herein, a “pharmaceutically acceptable carrier” refers to a non-toxic material that does not significantly interfere with the effectiveness of the biological activity of the active compound(s) to be administered, but is used as a composition ingredient, for example, to stabilize or protect the active compound(s) within a composition before use. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more active compounds of the invention are combined to facilitate application. The carrier may be co-mingled or otherwise mixed with one or more compositions as described herein, and/or with each other, in a manner such that there is no interaction which would substantially impair the desired pharmaceutical efficacy.

The compositions described herein may be administered in pharmaceutically acceptable compositions in some embodiments, which may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the active compound. For example, if the composition is a liquid, the carrier may be a solvent, partial solvent, or non-solvent, and may be aqueous or organically based. Non-limiting examples of suitable composition ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal.

Preparations include sterile aqueous or nonaqueous compositions, suspensions and emulsions, such as creams, gels, lotions, and the like. Non-limiting examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include, but are not limited to, alcoholic compositions, emulsions, or suspensions. Preservatives and other additives may also be present in some embodiments, such as, for example, antimicrobials, antioxidants, chelating agents, inert gases, and the like.

In some embodiments, a composition as described herein may be brought into association or contact with a suitable carrier, which may constitute one or more accessory ingredients. The final compositions may be prepared by any suitable technique, for example, by uniformly and intimately bringing a composition into association with a liquid carrier, a finely divided solid carrier, etc. optionally with one or more ingredients as previously described.

In some embodiments, the compositions may be prepared using processes such as hot melt extrusion, in which heat and pressure are applied to materials (e.g., pharmaceutical agents, eutectic solvents, such as deep eutectic solvents, polymers, excipients and other components such as those discussed herein) to cause them to melt and/or mix together. These are then extruded (e.g., through an orifice) and allowed to solidify. This may be done on a continuous basis in some embodiments. One example of such a process is OptiMelt® by Catalent.

The compositions as discussed herein, and optionally other therapeutics, may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. The term “pharmaceutically acceptable salts” includes salts of compositions described herein, prepared in combination with, for example, acids or bases. Pharmaceutically acceptable salts can be prepared as alkaline metal salts, such as lithium, sodium, or potassium salts; or as alkaline earth salts, such as beryllium, magnesium, or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like. Also, such salts can be prepared as alkaline metal or alkaline earth salts, such as sodium, potassium or calcium salts of the carboxylic acid group.

In another aspect, the present invention also provides any of the above-mentioned compositions in kits, optionally including instructions for use of the composition. In some cases, the kit can include a description of use of the compositions as discussed herein. The kit also can include instructions for use of a combination of two or more compositions. Instructions also may be provided for administering the compositions by any suitable technique as previously described, for example, orally, intravenously, pump or implantable delivery device, or via another known route of drug delivery.

The kits described herein may also contain one or more containers, which may contain compositions and other ingredients as previously described. The kits also may contain instructions for mixing, diluting, and/or administrating the compositions of the invention in some cases. The kits also can include other containers with one or more solvents, surfactants, preservatives, diluents, etc., as well as containers for mixing, diluting, or administering the components in a sample or to a subject in need of such treatment.

The compositions of the kit may be provided as any suitable form, for example, as a liquid. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In still another aspect, the invention includes the promotion of one or more of the above-described embodiments, e.g., in vitro or in vivo, e.g., by administering, to a subject, compositions such as those described herein. As used herein, “promoted” includes all methods of doing business, including methods of education, scientific inquiry, academic research, industry activity including pharmaceutical industry activity, and any advertising or other promotional activity including written, oral and electronic communication of any form.

Each of the following is incorporated herein by reference in its entirety: U.S. Provisional Patent Application Ser. No. 62/713,022, filed Aug. 1, 2018, entitled “Anhydrous Dosage Forms of Aspirin Based on Deep Eutectic Solvents and Other Specialized Media”; U.S. Provisional Patent Application Ser. No. 62/778,949, filed Dec. 13, 2018, entitled “Systems and Methods for Delivery of Drugs and Other Substances Comprising Deep Eutectic Solvents”; U.S. Provisional Patent Application Ser. No. 62/778,954, filed Dec. 13, 2018, entitled Methods and Systems Comprising Deep Eutectic Solvents for Poorly Soluble Drugs and Other Applications”; U.S. Provisional Patent Application Ser. No. 62/791,110, filed Jan. 11, 2019, entitled “Methods and Systems Comprising Deep Eutectic Solvents for Poorly Soluble Drugs and Other Applications”; U.S. Provisional Patent Application Ser. No. 62/817,065, filed Mar. 12, 2019, entitled “Eutectic Solvents Comprising Pharmaceutical Agents, and Methods of Making and Use Thereof”; and U.S. Provisional Patent Application Ser. No. 62/817,071, filed Mar. 12, 2019, entitled “Inhalable Eutectic Solvent Compositions and Methods.” In addition, a PCT application filed on even date herewith, entitled “Systems and Methods for Delivery of Drugs and Other Substances Comprising Deep Eutectic Solvents” is also incorporated herein by reference in its entirety.

The following examples are intended to illustrate certain embodiments of the present invention, but do not exemplify the full scope of the invention.

### Example 1

This example illustrates a composition, in accordance with one embodiment of the invention. The composition may or may not be anhydrous. First, a mixture of 1 part choline chloride to 2 parts urea by volume. That mixture was the mixed in a ratio of 1 part to 1 part propylene glycol. Next, 9 parts of that mixture was mixed with 1 part of citric acid. In one embodiment, this mixture was mixed with 25 mg/ml of loratadine. The mixture may thus be administered, e.g., as a liquid, for example in an inhaler. The effects of loratadine are expected to be much faster, as loratadine is normally relatively insoluble in water. In such a composition, loratadine would be administered, e.g., to the lungs, in a “pre-dissolved” state.

### Example 2

This example illustrates a composition, in accordance with another embodiment of the invention. The composition may or may not be anhydrous. The composition comprises a eutectic mixture of choline chloride and acetaminophen, where the choline chloride and acetaminophen form the eutectic. The eutectic may be contained within a capsule, e.g., for oral administration to a subject. The capsule contains about 500 mg of acetaminophen within the eutectic, and the volume of the eutectic within the capsule may be between 0.65 ml and 0.8 ml.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An composition, comprising:
a eutectic, wherein at least 20 mol % of the eutectic comprises choline chloride and acetaminophen.

2. The composition of claim 1, wherein at least 40 mol % of the eutectic comprises choline chloride and acetaminophen.

3. The composition of any one of claim 1 or 2, wherein at least 50 mol % of the eutectic comprises choline chloride and acetaminophen.

4. The composition of any one of claims 1-3, wherein at least 60 mol % of the eutectic comprises choline chloride and acetaminophen.

5. The composition of any one of claims 1-4, wherein at least 80 mol % of the eutectic comprises choline chloride and acetaminophen.

6. The composition of any one of claims 1-5, wherein the eutectic comprises less than 1 mol % water.

7. The composition of any one of claims 1-6, wherein the eutectic contains less than 0.1 mol % water.

8. The composition of any one of claims 1-7, wherein the eutectic contains less than 0.01 mol % water.

9. The composition of any one of claims 1-8, wherein the eutectic contains an undetectable amount of water.

10. The composition of any one of claims 1-9, wherein the eutectic is a liquid.

11. The composition of any one of claims 1-10, wherein the eutectic is a liquid at room temperature.

12. The composition of any one of claims 1-11, wherein the eutectic has a melting point that is less than 25° C.

13. The composition of any one of claims 1-12, wherein the eutectic has a melting point that is less than 20° C.

14. The composition of any one of claims 1-13, wherein the eutectic has a melting point that is less than 10° C.

15. The composition of any one of claims 1-14, wherein the acetaminophen comprises at least 30 mol % of the eutectic.

16. The composition of any one of claims 1-15, wherein the acetaminophen comprises at least 40 mol % of the eutectic.

17. The composition of any one of claims 1-16, wherein the acetaminophen comprises at least 45 mol % of the eutectic.

18. The composition of any one of claims 1-17, wherein the choline chloride comprises at least 30 mol % of the eutectic.

19. The composition of any one of claims 1-18, wherein the choline chloride comprises at least 40 mol % of the eutectic.

20. The composition of any one of claims 1-19, wherein the choline chloride comprises at least 45 mol % of the eutectic.

21. The composition of any one of claims 1-20, wherein the choline chloride and the acetaminophen are a present within the eutectic at a ratio that causes the eutectic to have a melting point of less than 25° C.

22. The composition of any one of claims 1-21, wherein the choline chloride and acetaminophen are a present within the eutectic at a ratio that causes the eutectic to have a melting point of less than 20° C.

23. The composition of any one of claims 1-22, wherein at least 90 mol % of the eutectic comprises choline chloride and acetaminophen.

24. The composition of any one of claims 1-23, wherein at least 95 mol % of the eutectic comprises choline chloride and acetaminophen.

25. The composition of any one of claims 1-24, wherein the eutectic consists essentially of choline chloride, and acetaminophen.

26. The composition of any one of claims 1-25, wherein the acetaminophen is present within the composition such that the concentration of choline chloride and acetaminophen is at least 1.25 g/ml.

27. The composition of any one of claims 1-26, wherein the acetaminophen is present within the composition such that the concentration of choline chloride and acetaminophen is at least 1.3 g/ml.

28. The composition of any one of claims 1-27, wherein the eutectic is contained within a capsule.

29. The composition of claim 28, wherein the capsule contains no more than 1000 mg of acetaminophen.

30. The composition of any one of claim 28 or 29, wherein the capsule contains no more than 750 mg of acetaminophen.

31. The composition of any one of claims 28-30, wherein the capsule contains between 400 mg and 600 mg of acetaminophen.

32. The composition of any one of claims 28-31, wherein the capsule has an inner volume of no more than 1 ml.

33. The composition of any one of claims 28-31, wherein the capsule has an inner volume of no more than 0.8 ml.

34. A method, comprising orally administering the composition of any one of claims 1-33 to a subject.

35. The method of claim 34, wherein the subject is human.

36. A method, comprising:
orally administering a composition to a subject, wherein the composition comprises a eutectic, wherein at least 20 mol % of the eutectic comprises choline chloride and acetaminophen.

37. A composition, comprising:
a eutectic, wherein at least 20 mol % of the eutectic comprises choline chloride and a pharmaceutical agent comprising a hydrogen bond donor.

38. The composition of claim 37, wherein at least 40 mol % of the eutectic comprises choline chloride and the pharmaceutical agent.

39. The composition of any one of claim 37 or 38, wherein at least 50 mol % of the eutectic comprises choline chloride and the pharmaceutical agent.

40. The composition of any one of claims 37-39, wherein at least 60 mol % of the eutectic comprises choline chloride and the pharmaceutical agent.

41. The composition of any one of claims 37-40, wherein at least 80 mol % of the eutectic comprises choline chloride and the pharmaceutical agent.

42. The composition of any one of claims 37-41, wherein the eutectic comprises less than 1 mol % water.

43. The composition of any one of claims 37-42, wherein the eutectic contains less than 0.1 mol % water.

44. The composition of any one of claims 37-43, wherein the eutectic contains less than 0.01 mol % water.

45. The composition of any one of claims 37-44, wherein the eutectic contains an undetectable amount of water.

46. The composition of any one of claims 37-45, wherein the eutectic is a liquid.

47. The composition of any one of claims 37-46, wherein the eutectic is a liquid at room temperature.

48. The composition of any one of claims 37-47, wherein the eutectic has a melting point that is less than 25° C.

49. The composition of any one of claims 37-48, wherein the eutectic has a melting point that is less than 20° C.

50. The composition of any one of claims 37-49, wherein the eutectic has a melting point that is less than 10° C.

51. The composition of any one of claims 37-50, wherein the pharmaceutical agent comprises at least 30 mol % of the eutectic.

52. The composition of any one of claims 37-51, wherein the pharmaceutical agent comprises at least 40 mol % of the eutectic.

53. The composition of any one of claims 37-52, wherein the pharmaceutical agent comprises at least 45 mol % of the eutectic.

54. The composition of any one of claims 37-53, wherein the choline chloride comprises at least 30 mol % of the eutectic.

55. The composition of any one of claims 37-54, wherein the choline chloride comprises at least 40 mol % of the eutectic.

56. The composition of any one of claims 37-55, wherein the choline chloride comprises at least 45 mol % of the eutectic.

57. The composition of any one of claims 37-56, wherein the choline chloride and the pharmaceutical agent are a present within the eutectic at a ratio that causes the eutectic to have a melting point of less than 25° C.

58. The composition of any one of claims 37-57, wherein the choline chloride and the pharmaceutical agent are a present within the eutectic at a ratio that causes the eutectic to have a melting point of less than 20° C.

59. The composition of any one of claims 37-58, wherein at least 90 mol % of the eutectic comprises choline chloride and the pharmaceutical agent.

60. The composition of any one of claims 37-599, wherein at least 95 mol % of the eutectic comprises choline chloride and the pharmaceutical agent.

61. The composition of any one of claims 37-60, wherein the eutectic consists essentially of choline chloride, and the pharmaceutical agent.

62. The composition of any one of claims 37-61, wherein the pharmaceutical agent is a H-bond donor.

63. The composition of any one of claims 37-62, wherein the pharmaceutical agent comprises acetaminophen.

64. The composition of any one of claims 37-63, wherein the pharmaceutical agent is present within the composition such that the concentration of choline chloride and acetaminophen is at least 1.25 g/ml.

65. The composition of any one of claims 37-64, wherein the pharmaceutical agent is present within the composition such that the concentration of choline chloride and acetaminophen is at least 1.3 g/ml.

66. The composition of any one of claims 37-65, wherein the eutectic is contained within a capsule.

67. The composition of claim 66, wherein the capsule contains no more than 1000 mg of the pharmaceutical agent.

68. The composition of any one of claim 66 or 67, wherein the capsule contains no more than 750 mg of pharmaceutical agent.

69. The composition of any one of claims 66-68, wherein the capsule contains between 400 mg and 600 mg of pharmaceutical agent.

70. The composition of any one of claims 66-69, wherein the capsule has an inner volume of no more than 1 ml.

71. The composition of any one of claims 66-70, wherein the capsule has an inner volume of no more than 0.8 ml.

72. A method, comprising orally administering the composition of any one of claims 37-71 to a subject.

73. The method of claim 72, wherein the subject is human.

74. A method, comprising:
orally administering a composition to a subject, wherein the composition comprises a eutectic composition, wherein at least 20 mol % of the eutectic composition comprises choline chloride and a pharmaceutical agent comprising a hydrogen bond donor.

75. A method, comprising:
administering a composition comprising a pharmaceutically acceptable deep eutectic solvent into the lungs of a subject, wherein the deep eutectic solvent comprises a pharmaceutical agent dissolved therein.

76. The method of claim 75, wherein the deep eutectic solvent comprises choline chloride and urea.

77. The method of claim 76, wherein the choline chloride is present in the deep eutectic solvent at between 25 mol % and 75 mol %.

78. The method of any one of claim 76 or 77, wherein the urea is present in the deep eutectic solvent at between 25 mol % and 75 mol %.

79. The method of any one of claims 76-78, wherein the choline chloride is present in the deep eutectic solvent at between 40 mol % and 60 mol %.

80. The method of any one of claims 76-79, wherein the urea is present in the deep eutectic solvent at between 40 mol % and 60 mol %.

81. The method of any one of claims 76-80, wherein the choline chloride and the urea are present within the deep eutectic solvent at a ratio that causes the deep eutectic solvent to have a melting point of less than 25° C.

82. The method of any one of claims 76-81, wherein the choline chloride and the urea are a present within the deep eutectic solvent at a ratio that causes the deep eutectic solvent to have a melting point of less than 20° C.

83. The method of any one of claims 75-82, wherein the composition comprises at least 80 mol % of the deep eutectic solvent.

84. The method of any one of claims 75-83, wherein the composition comprises at least 90 mol % of the deep eutectic solvent.

85. The method of any one of claims 75-84, wherein the composition comprises at least 95 mol % of the deep eutectic solvent.

86. The method of any one of claims 75-85, wherein the pharmaceutical agent comprises fluticasone.

87. The method of any one of claims 75-86, wherein the pharmaceutical agent comprises loratadine.

88. The method of any one of claims 75-87, wherein the pharmaceutical agent comprises cetirizine.

89. The method of any one of claims 75-88, wherein the pharmaceutical agent comprises insulin.

90. The method of any one of claims 75-89, wherein the pharmaceutical agent comprises sumatriptan.

91. The method of any one of claims 75-90, wherein the pharmaceutical agent comprises an antihistamine.

92. The method of any one of claims 75-91, comprising administering the deep eutectic solvent using an inhaler.

93. The method of any one of claims 75-92, wherein the deep eutectic solvent is administered to the lungs as droplets.

94. The method of any one of claims 75-93, wherein at least some of the droplets administered to the lungs are suspended in air.

95. The method of any one of claims 75-94, wherein the droplets have an average diameter of less than about 100 micrometers.

96. The method of any one of claims 75-95, wherein the droplets have an average diameter of less than about 10 micrometers.

97. The method of any one of claims 75-96, wherein the deep eutectic solvent contains less than 1 mol % water.

98. The method of any one of claims 75-97, wherein the deep eutectic solvent contains less than 0.1 mol % water.

99. The method of any one of claims 75-98, wherein the deep eutectic solvent contains an undetectable amount of water.

100. The method of any one of claims 75-99, wherein the deep eutectic solvent is a liquid at room temperature.

101. The method of any one of claims 75-100, wherein the deep eutectic solvent has a melting point that is less than 25° C.

102. The method of any one of claims 75-101, wherein the deep eutectic solvent has a melting point that is less than 20° C.

103. The method of any one of claims 75-102, wherein the deep eutectic solvent has a melting point that is less than 10° C.

104. The method of any one of claims 75-103, wherein the subject is human.

105. An article, comprising:
an inhaler comprising a pharmaceutically acceptable deep eutectic solvent comprising a pharmaceutical agent.

106. The article of claim 105, wherein the deep eutectic solvent comprises choline chloride and urea.

107. The article of claim 106, wherein the choline chloride is present in the deep eutectic solvent at between 25 mol % and 75 mol %.

108. The article of any one of claim 106 or 107, wherein the urea is present in the deep eutectic solvent at between 25 mol % and 75 mol %.

109. The article of any one of claims 106-108, wherein the choline chloride and the urea are present within the deep eutectic solvent at a ratio that causes the deep eutectic solvent to have a melting point of less than 25° C.

110. The article of any one of claims 105-109, wherein the composition comprises at least 80 mol % of the deep eutectic solvent.

111. The article of any one of claims 105-110, wherein the pharmaceutical agent comprises fluticasone.

112. The article of any one of claims 105-111, wherein the pharmaceutical agent comprises loratadine.

113. The article of any one of claims 105-112, wherein the pharmaceutical agent comprises cetirizine.

114. The article of any one of claims 105-113, wherein the pharmaceutical agent comprises insulin.

115. The article of any one of claims 105-114, wherein the pharmaceutical agent comprises sumatriptan.

116. The article of any one of claims 105-115, wherein the pharmaceutical agent comprises an antihistamines.

117. The article of any one of claims 105-116, wherein the deep eutectic solvent contains less than 1 mol % water.

118. The article of any one of claims 105-117, wherein the deep eutectic solvent is a liquid at room temperature.

119. The article of any one of claims 105-118, wherein the deep eutectic solvent has a melting point that is less than 25° C.

120. A method, comprising using the inhaler of any one of claims 105-119 to administer the deep eutectic solvent to the lungs of a subject.
